(19) United States
(12) Patent Application Publication
(10) Pub. No.: US 2009/0104669 A1
(43) Pub. Date: Apr. 23, 2009

(54) Method for Preparing (S)-3-Hydroxy-Gamma-Butyrolactone Using Hydrolase

(76) Inventors: Oh-Jin Park, Daejeon (KR); Sang-Hyun Lee, Daejeon (KR); Sang-Who Lee, Daejeon (KR); Goon-Ho Joe, Daejeon (KR); Hong-Sun Uh, Seoul (KR)

Correspondence Address: MCKENNA LONG & ALDRIDGE LLP, 1900 K STREET, NW, WASHINGTON, DC, 20006, US

(21) Appl. No.: 12/224,548

(22) PCT Filed: Mar. 2, 2007
(86) PCT No.: PCT/KR2007/001052

§ 371 Date: Aug. 29, 2008

(30) Foreign Application Priority Data
Mar. 2, 2006 (KR) 10 2006 0019944

(51) Int. Cl. C12P 17/04

(52) U.S. Cl. 435/126

(57) ABSTRACT

The present invention relates to a method for preparing S-HGB ((S)-3-hydroxy-γ-butyrolactone) using hydrolase, and more particularly to a method for preparing S-HGB in a high purity by hydrolyzing S-BBL ((S)-β-benzoyloxy-γ-butyrolactone) in the presence of hydrolase. According to the present invention, the S-HGB having an optical purity can be obtained in a high yield under simple process conditions without requiring reaction conditions of high pressure and high temperature or complex operating conditions by hydrolyzing S-BBL with hydrolase.

### TECHNICAL FIELD

The present invention relates to a method for preparing high-purity S-HGB ((S)-3-hydroxy-γ-butyrolactone) by hydrolyzing S-BBL ((S)-β-benzoyloxy-γ-butyrolactone).

### BACKGROUND ART

S-HGB ((S)-3-hydroxy-γ-butyrolactone) is a chiral compound having asymmetric carbon atoms, and has been used in the various fields of applications. For example, the S-HGB was used as an intermediate for manufacturing Lipitor® (Atorvastatin, Pfizer) and Zyvox® (Linezolid, Pharmacia & Upjohn), and used also as an ingredient, L-Carnitine, of food additives.

The methods for synthesizing S-HGB have been reported in a large number of patents and literatures. For example, it was reported that L-malic acid is selectively reduced in the presence of catalyst, in particular the presence of sodium borohydride and metal catalyst in U.S. Pat. No. 5,808,107 and U.S. Pat. No. 6,429,319, respectively. However, these methods have problems that their operating conditions are very complicated and expensive apparatuses are required since the methods are continuously carried out at high temperature and high pressure (50-550° C., 15-5,500 psig).

The synthesis of S-HGB in the reduction reaction of β-ketoester using an enzyme or catalyst (J. Am. Chem. Soc., 105:5925, 1983) has problems that optical purity of the S-HGB is low and a used metal catalyst is expensive.

Also, there has been also reported a method for producing S-HGB in the dehalogenation reaction using a microorganism (Enterobacter Sp. DS-S-75) (Suzuki et al., Enzyme Microb. Technol., 24:13, 1999), but the method has problems that the S-HGB has a low yield and optical purity.

There are some reports on methods for synthesizing S-HGB in the oxidation reaction using inexpensive soluble carbohydrate as a starting material (U.S. Pat. No. 5,292,939; U.S. Pat. No. 5,374,773; and U.S. Pat. No. 6,124,122), but these methods have a disadvantage that a yield of the S-HGB is low since they are exothermic and use harmful compounds such as sulfuric acid, and it is difficult to separate and purify the final product.

As described above, the conventional methods for producing S-HGB have problems that their processes are complicated, an optical purity of the obtained S-HGB is low, and the used reagents are dangerous in their handling and harmful to environments. Accordingly, there are limitations on their commercialization since the complex processes causes reduction in yield of the S-HGB.

Meanwhile, in U.S. Pat. No. 5,928,933, it was reported that only one of forty-four protease, lipase and esterase enzymes shows an optical purity of 95% when a method for kinetically resolving enantiomers in the selective hydrolysis using an enzyme is selected to produce a chiral compound having asymmetric carbon atoms, enantioselectivities of hydrolase is used to confirm reaction specificities against the forty-four enzymes. In the synthesis of vitamin precursor using regioselectivity of an enzyme (Gotor et al., J. Org. Chem. 67:1266, 2002; and EP 1,239,045), there is reported a method for hydrolyzing one of two esters, which are present in one molecule, by using hydrolase.

Also, Japanese Patent Publication No. 2003-299496 discloses a method for producing S-HGB using microorganism-derived esterase, but the method has difficulty in obtaining S-HGB in a high yield since the S-HGB is produced by reducing 4-halo-3-oxo butane acid ester through a two-step process.

### DISCLOSURE

### Technical Problem

Accordingly, the inventors have made comprehensive studies on the method for producing S-HGB, and therefore they found that S-HGB having a high optical purity may be obtained in a high yield while suppressing side reactions by hydrolyzing S-BBL with hydrolase. As a result, the present invention is completed according to the above findings.

Accordingly, it is an object of the present invention to provide a method for preparing S-HGB having a high optical purity (ee >99.5%) by hydrolyzing S-BBL in the presence of hydrolase.

### Technical Solution

In order to accomplish the above object, the present invention provides a method for preparing S-HGB ((S)-3-hydroxy-γ-butyrolactone), wherein the S-HGB is obtained by hydrolyzing (S)-β-benzoyloxy-γ-butyrolactone (S-BBL) represented by the following Formula 1 in the presence of hydrolase:

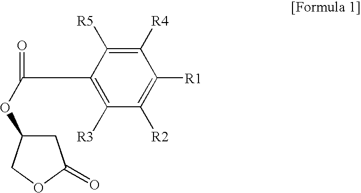

in the Formula 1,

R1, R2, R3, R4 and R5 are independently one selected from the group consisting of hydrogen, (C1-C10)alkyl, (C5-C6)cycloalkyl, fluoro(C1-C10)alkyl, OR′, aryl, aryl(C1-C10)alkyl, NO₂, NR′R″, C(O)R′, CO₂R′, C(O)NR′R″, N(R″)C(O)R′, N(R″)CO₂R′, N(R″)C(O)NR′R″, S(O)ₘNR′R″, S(O)ₘR′, CN and N(R″)S(O)ₘR′, provided that two adjacent Rs among the groups R1, R2, R3, R4 and R5 may be an aromatic or cycloalkene ring fused by sharing carbon atoms,

the R′ and R″ are independently one selected from the group consisting of hydrogen, (C1-C10)alkyl, aryl and aryl(C1-C10)alkyl, provided that the R′ and R″ may be a 5-, 6- or 7-membered ring containing 1 to 3 heteroatoms selected from the group consisting of N, O and S since the R′ and R″ are binds to each other if they are connected to the same nitrogen atom, and

m is an integer from 0 to 2.

In the present invention, it may be characterized in that the hydrolysis of S-BBL is carried out in a hydrophilic solvent or a two-phase system of a hydrophilic solvent-hydrophobic organic solvent. As the hydrophilic solvent, it is preferred to use at least one selected from the group consisting of water, lower alcohol including methanol, ethanol, propan-2-ol, etc., acetic acid, acetone, ethylacetate, acetonitrile, N,N-dimethylformamide, N,N-dimethylacetamide, dimethylsulfoxide, pyridine, tetrahydrofuran, dioxane, dimethylformamide and dimethylsulfoxide, and as the hydrophobic organic solvent, it is preferred to use at least one selected from the group consisting of isopropyl ether, tert-butyl methyl ether (TBME), chloroform, dichloromethane, carbon tetrachloride, hexane, toluene and cyclohexane.

In the present invention, it may be characterized in that the hydrolase is selected from the group consisting of lipase, proteinase and esterase, and the hydrolase is particularly preferably Candida rugosa-derived lipase, Alcaligenes sp.-derived lipase or their mixture.

The present invention also provides a method for preparing high-purity S-HGB, the method comprising (a) preparing S-HGB by hydrolyzing S-BBL in the presence of hydrolase: (b) removing enzymes from the hydrolysate of the step (a) and extracting benzoic acid, which is present as a by-product in the resultant product, with a hydrophobic organic solvent; and (c) obtaining S-HGB by extracting the benzoic acid-free hydrolysate of the step (b) with a hydrophilic organic solvent at 0° C. or below.

In the method, it may be characterized in that the enzyme of the step (b) is removed using a filtration method. Also, as the hydrophobic organic solvent for removing benzoic acid in the step (b), it is preferred to use at least one selected from the group consisting of isopropyl ether, tert-butyl methyl ether (TBME), chloroform, dichloromethane, carbon tetrachloride, hexane, toluene and cyclohexane. As the hydrophilic organic solvent for extracting S-HGB in the step (c), it is preferred to use at least one selected from the group consisting of lower alcohol including methanol, ethanol and propan-2-ol, acetic acid, acetone, ethylacetate, acetonitrile, N,N-dimethylformamide, N,N-dimethylacetamide, dimethylsulfoxide, pyridine, tetrahydrofuran, dioxane, dimethylformamide and dimethylsulfoxide.

Hereinafter, the present invention will be described in more detail, as follows.

At first, the method of the present invention includes a step of preparing S-BBL represented by the Formula 1. The S-BBL represented by the Formula 1 may be produced using the following method, but the present invention is not limited to the following exemplary methods for preparing certain S-BBL. For example, (S)-β-benzoyloxy-γ-butyrolactone in which R1 to R5 in the S-BBL represented by the Formula 1 are all hydrogen may be synthesized by synthesizing S-BSA ((S)-2-benzoyloxy-succinic anhydride) from benzoil chloride and L-malic acid, followed by reacting the S-BSA with ZnCl₂/KBH₄. The method for preparing S-BBL is described in detail in Korean Patent Publication No. 2002-0073751.

Next, S-HGB is produced by hydrolyzing the S-BBL with a hydrolase powder or an immobilized enzyme in a hydrophilic solvent or a two-phase system of hydrophilic solvent-hydrophobic organic solvent which is maintained at constant pH and temperature. At this time, the used hydrophilic solvent is preferably at least one selected from the group consisting of water, lower alcohol including methanol, ethanol and propan-2-ol, acetic acid, acetone, ethylacetate, acetonitrile, N,N-dimethylformamide, N,N-dimethylacetamide, dimethylsulfoxide, pyridine, tetrahydrofuran, dioxane, dimethylformamide and dimethylsulfoxide, and the used hydrophobic organic solvent is preferably at least one selected from the group consisting of isopropyl ether, tert-butyl methyl ether (TBME), chloroform, dichloromethane, carbon tetrachloride, hexane, toluene and cyclohexane. The organic solvent is more preferably TBME, cyclohexane, hexane and mixture thereof, which may stably sustain activity of the enzyme. After the production of the S-HGB is completed, the used enzyme is removed by filtration, and a by-product, benzoic acid, produced along with the S-HGB is extracted with a hydrophobic organic solvent. At this time, the S-HGB remaining in an aqueous solution is collected by evaporation of the solvent and extraction of the hydrophilic (polar) organic solvent. The used hydrophilic solvent and hydrophobic organic solvent are the same solvent used in the hydrolyzation.

The S-HGB, collected as described above, has a very high optical purity (for example, ee >99.5%). Particularly, the immobilized enzyme has an advantage that it may be re-used since it is collected from the reaction products through the filtration.

The hydrolase used in the present invention may be selected from at least one selected from the group consisting of lipase, proteinase and esterase, and the enzyme may be in a form of powder or an aqueous solution. In some cases, the enzyme immobilized on a carrier may also be used. As the method for immobilizing an enzyme, various methods are known as apparent to those skilled in the art, for example, by attaching an enzyme to a polymer carrier or an inorganic carrier such as celite.

Amount of the used enzyme may be determined according to reaction temperature, pH, amount of reactants, reaction time, etc., and therefore an effective amount of the enzyme is varied, and preferably about 0.1˜100% by weigh, based on the weight of a substrate. In this case, a reaction time is extended if the amount of the used enzyme is too small, while the use of the excessive enzyme results in inadequate problems in the reaction process such as low economical efficiency and difficulty in separation of the enzyme if the amount of the enzyme is used in a large amount.

There is no particular limitation on the reaction conditions using the hydrolase, but the reaction conditions is a temperature of 0˜60° C. and a pH value of 3˜12 so as to optimize the enzyme reaction. The reaction temperature is more preferably 30˜50° C. As described above, the enzyme reaction may be carried out in an aqueous solution (a hydrophilic solvent), and a two-phase system of an organic solvent-aqueous solution (a hydrophilic solvent) mixture may be used in a small amount to enhance solubility of the substrate and reduce an inhibitory effect of products on the enzyme.

Concentration of the substrate used for the enzyme reaction may also be varied according to a variety of reaction factors, and is preferably 500 mM˜1 M to optimize the enzyme reaction, but it is possible to use a higher concentration of the substrate. In the case of the reaction in the high concentration of the enzyme, an ester compound is not easily dissolved at the beginning of the reaction, but the reaction is accelerated by contact of the enzyme with the ester compound dissolved in water. In order to increase a reaction rate of the enzyme and maintain activity of the enzyme, the reaction may be carried out in a solvent containing the minimum amount of water and an organic solvent as a main component.

The optimum conditions of the enzyme reaction may be determined in consideration of other various reaction factors, in addition to the factors as described above.

In order to prepare S-HGB in a high purity according to the present invention, the S-HGB prepared in the hydrolyzation is extracted with an organic solvent at a low temperature of 0° C. or below, which is to prevent deterioration in the S-HGB.

### DESCRIPTION OF DRAWINGS

FIG. 1 is a diagram showing a chromatographic analysis of a purity of S-BBL prepared according to one embodiment of the present invention.

FIG. 2 is a diagram showing a chromatographic analysis of a S-BBL hydrolysate using a free enzyme (A: 1.5 hours, B: 2 hours).

FIG. 3 is a diagram showing a chromatographic analysis of a S-BBL hydrolysate using an immobilized enzyme (A: 38 hours, B: 60 hours).

### BEST MODEL

Hereinafter, preferred embodiments of the present invention will be described in detail referring to the accompanying drawings. However, the description proposed herein is just a preferable example for the purpose of illustrations only, not intended to limit the scope of the invention, so it should be understood that other equivalents and modifications could be made thereto without departing from the spirit and scope of the invention. The preferred embodiments of the present invention will be described in detail for the purpose of better understandings, as apparent to those skilled in the art.

### EXAMPLE 1

### Synthesis of S-BBL

1.5M benzoil chloride and 0.5M L-malic acid were reacted at 60° C. for 10 hours, and washed with TBME to prepare S-BSA ((S)-2-benzoyloxy-succinic anhydride). One equivalent of S-BSA and 0.5 equivalent of Zn (BH₄)₂ were added to THF (tetrahydrofuran), reacted at a constant temperature of 25° C. for 5 hours while stirring, and then S-BBL was prepared by evaporating THF from the resultant mixture and extracting the mixture with dichloromethane.

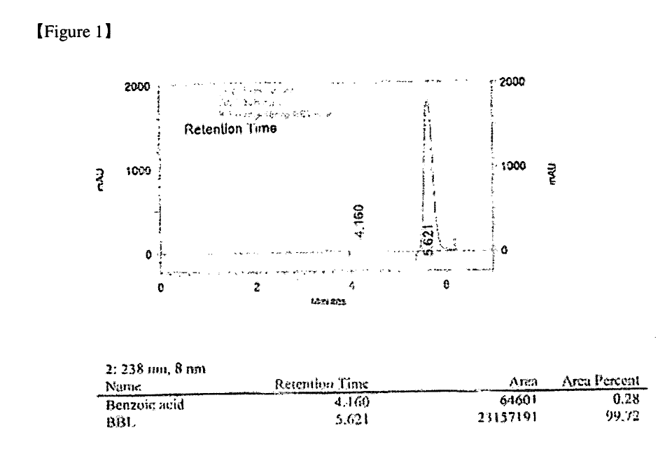

HPLC analysis of the prepared S-BBL was shown in FIG. 1. As shown in FIG. 1, a retention time of the S-BBL was 5.6 minutes, and its purity was 99.7%. Accordingly, it was revealed that the S-BBL obtained according to the method has a purity of 99% or more.

Conditions of the HPLC analysis is as follows: acetonitrile:water=60:40 (v/v), C18 Capcell Pak, 1 ml/min, 238 nm UV).

### EXAMPLE 2

### Screening of S-BBL Hydrolase

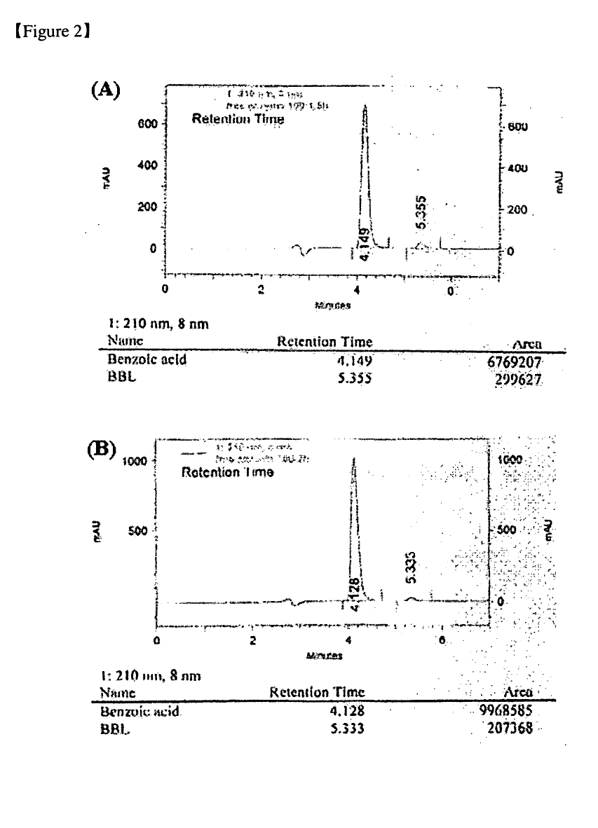

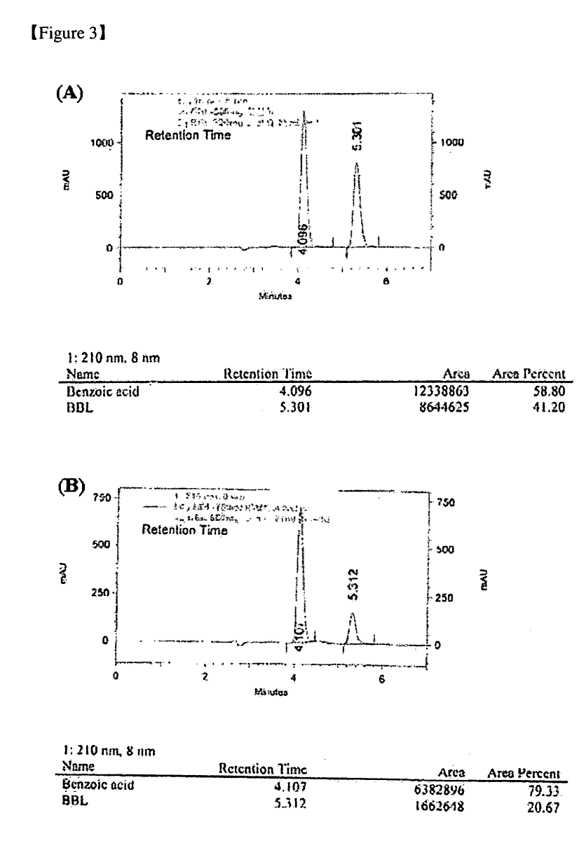

10 mg of S-BBL and 17 mg of enzyme powder were added to a mixture solution of 250 ul of Tris-HCl buffer (0.1M, pH 7.0) and 200 ul of acetone, reacted at 37° C. for 24 hours and 48 hours, and then the resultant test samples were taken and hydrolyzed under the HPLC condition of the Example 1, and the resultant product was analyzed (benzoic acid: 4.1 min, S-BBL: 5.3 min). The benzoic acid was a hydrolysate by-product, and the S-BBL was a remaining starting material which remains unhydrolyzed. The results are shown in FIGS. 2 and 3.

FIGS. 2 and 3 are diagrams showing chromatographic analysis profiles of S-BBL hydrolysates according to the reaction time, the hydrolysates being obtained by using a free enzyme and an immobilized enzyme, respectively. FIGS. 2 (A) and (B) show analysis profiles of the S-BBL hydrolysates when the reaction times are 1.5 hours and 2 hours, respectively, and FIGS. 3 (A) and (B) show analysis profiles of the S-BBL hydrolysates when the reaction times are 38 hours and 60 hours, respectively.

At this time, the generated S-HGB in the reaction product was not observed in an ultraviolet (UV), and therefore observed by development in an iodine chamber.

The kinds and conversions of the enzymes used in this Example were listed in the following Table 1. The S-BBL was converted into the S-HGB by various kinds of hydrolases, and the conversion is the most excellent if Candida rugosa- and Alcaligenes sp.-derived lipases are used herein.

TABLE 1

| | | | Conversion (%) | |
|---|---|---|---|---|
| Enzyme | | Source | 1 Day | 2 Days |
| Lipase | Lipase PS | Burkholderia cepacia | 16 | 22 |
| | Novozym 525L | Candida antarctica | 20 | 29 |
| | Lipase OF | Candida rugosa | 100 | — |
| | Lipase PL | Alcaligenes sp. | 100 | — |
| | Lipase PS-D | Burkholderia cepacia | 13 | 18 |
| | Lipase PS-C | Burkholderia cepacia | — | 56 |
| | Lipase MY | Candida rugosa | 49.5 | 23 |
| | Lipase AYS | Candida rugosa | 66 | 81 |
| | Lipozyme | Humicola lanuginosa | 17 | 28 |
| | Lipase (Sigma | Candida rugosa. | 47 | 59 |
| | type VII) | | | |
| | Lipoprotein | Pseudomonas sp. | 44 | 50 |
| | lipase (Toyobo) | | | |
| | Lipase AH | Burkholderia cepacia | 31 | 46 |
| | Novozym 525L | Candida antarctica | 20 | 29 |
| Proteinase | Protease PS | Bacillus sp. | 23 | 32 |
| | Neutral | Bacillus | 12 | 16 |
| | Proteinase | | | |
| | (Toyobo) | | | |
| Esterase | PLE-AL amano | Porcine liver | 21 | 33 |
| | PLE-Roche | Porcine liver | 28 | 34 |
| Others | Alcalase 0.6 L | Bacillus licheniformis | 35 | 48 |
| | Alcalase (0.6 L) | Bacillus licheniformis | 21 | 26 |
| | Acylase Amano | Aspergillus melleus | 47 | 59 |
| | PGA-450 (Roche) | E. coli | 61 | 83 |

### EXAMPLE 3

### Effect of Organic Solvent on Enzyme Reaction

10 mg of S-BBL and 10 mg of Lipase OF enzyme powder were added to a mixture solution of 100 ul of Tris-HCl buffer (0.1M, pH 7.0) and 300 ul of acetonitrile, acetone and TBME, reacted at 37° C. for 2 hours and 6 hours while stirring at a rotary speed of 200 rpm, and then the resultant test samples were taken in a dose of 20 ul and diluted with 980 ul of acetonitrile, and then the resultant product was analyzed under the HPLC conditions of Example 1. The results are listed in the following Table 2.

TABLE 2

Conversion (%)

| Organic Solvent | 30 Min | 60 Min |
|---|---|---|
| TBME | 100 | — |
| Acetone | 44 | 73 |
| Acetonitrile | <1 | <1 |

As a result, the reaction solvent of the enzyme for preparing S-HGB more preferably includes non-polar TBME than acetone or acetonitrile having polarity since the non-polar TBME may stably maintain activity of the enzyme, as listed in the Table 2.

### EXAMPLE 4

### Change in Relative Amount of Enzyme and S-BBL

The method of Example 3 was repeated to analyze the final product every 30 minutes with varying amounts of the Lipase OF and the substrate S-BBL. The results are listed in the following Table 3.

TABLE 3

Conversion (%)

| S-BBL:Lipase OF | 30 Min | 60 Min |
|---|---|---|
| 20:1 | 41.3 | 65.8 |
| 20:2 | 64.4 | 75 |
| 20:3 | 87.5 | 100 |
| 20:4 | 100 | — |

As a result, it was revealed that the conversion from S-BBL to S-HGB is increased to a certain level as an amount of the enzyme increases when the substrate is used in an equivalent amount, as listed in the Table 3.

### EXAMPLE 5

### Immobilization and Re-Use of Enzyme

Amberlite® XAD-7 supplied from Rohm & Haas, which is polymethacrylate-based cross-linked polymer particle having a mesh size of 20˜60, was used. 1 g of Lipase OF (Meito Sagyo, Japan) was dissolved at a room temperature in 50 mL of 1M phosphate buffer (pH 6.0), 0.5 g and 1 g of XAD-7 was added, respectively, to 2.5 mL of a solution (equivalent to 50 mg of the enzyme), mixed, and then stirred overnight to bind the XAD-7 to the substrate. The bound enzyme was collected by filtration, and then activity of the enzyme in the supernatant was measured. As a result, the XAD-7-bound resin showed an activity of 5%, compared to the XAD-7-free Lipase OF.

The enzyme-bound resin was put into 5 mL of 0.1M phosphate buffer (pH 7.0), and 25 ul of 25% glutaraldehyde solution was added, and then treated at a room temperature for 6 hours. After filtration, 1.5 g of S-BBL was added to a mixture of 7.5 mL of water and 7.5 mL of TBME, and then reacted at 35° C. while stirring. The immobilized enzyme was collected by filtering the reaction solution, and then re-used in a repeated manner in which 1.5 g of S-BBL was added to a mixture of 7.5 mL of water and 7.5 mL of TBME and reacted at 35° C. while stirring. The results are listed in the following Table 4.

TABLE 4

| | |
|---|---|
| Reaction Time | Conversion (%) |

| Use Count | (hr) | 0.5 g XAD-7 | 1 g XAD-7 |
|---|---|---|---|
| 1 | 20 | 95 | 86 |
| 2 | 21 | 91 | 88 |
| 3 | 20 | 89 | 79 |
| 4 | 38 | 99 | 96.3 |
| 5 | 22 | 71 | 72 |
| 6 | 27 | 81 | 83 |
| 7 | 23 | 68 | 50 |

As listed in Table 4, it was revealed that the activity of the enzyme is not lowered even after the enzyme was re-used 6 times.

### EXAMPLE 6

### Separation and Purification of S-HGB

1 kg of Lipase OF was immobilized onto 5 kg of XAD-7 in the same manner as in Example 5, added to a mixture solution of 100 L of water and 100 L of TBME, and then 20.5 kg of S-BBL was added and reacted for 11 hours, and a TBME fraction was removed.

20.5 kg of S-BBL and 100 L of TBME were newly added to the reaction solution from which the TBME fraction was removed, reacted for 12 hours, and then a TBME fraction was removed. Also, 20.5 kg of S-BBL and 100 L of TBME were additionally added to the reaction solution, reacted for 14 hours, and then a TBME fraction was removed. Subsequently, an immobilized enzyme was removed by filtering an aqueous solution fraction, and a reaction product was washed with TBME to remove a by-product, benzoic acid, and then a solution, obtained by washing the reaction product with 100 ml of a polar solvent, ethyl acetate, three times at 0° C. or below, was distilled at 30° C. under a reduced pressure.

The reaction product was extracted using the method as described above to obtain 24.3 kg of S-HGB having a purity of 99%.

As described above, the specific embodiments of the present invention has been described in detail. However, the description proposed herein is just a preferable example for the purpose of illustrations only, not intended to limit the scope of the invention, so it should be understood that other equivalents and modifications could be made thereto without departing from the spirit and scope of the invention, as apparent to those skilled in the art.

### INDUSTRIAL APPLICABILITY

According to the present invention, the S-HGB ((S)-3-hydroxy-γ-butyrolactone) having an optical purity can be obtained in a high yield under simple process conditions without requiring reaction conditions of high pressure and high temperature or complex operating conditions by hydrolyzing S-BBL ((S)-β-benzoyloxy-γ-butyrolactone) with hydrolase.

1. A method for preparing (S)-3-hydroxy-γ-butyrolactone (S-HGB), wherein the S-HGB is obtained by hydrolyzing (S)-β-benzoyloxy-γ-butyrolactone (S-BBL) represented by the following Formula 1 in the presence of hydrolase: 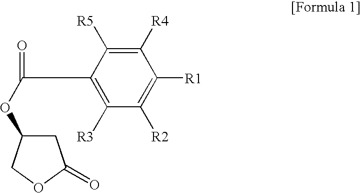
in the Formula 1,
R1, R2, R3, R4 and R5 are independently one selected from the group consisting of hydrogen, (C1-C10)alkyl, (C5-C6)cycloalkyl, fluoro(C1-C10)alkyl, OR′, aryl, aryl(C1-C10)alkyl, NO₂, NR′R″, C(O)R′, CO₂R′, C(O)NR′R″, N(R″)C(O)R′, N(R″)CO₂R′, N(R″)C(O)NR′R″, S(O)ₘNR′R″, S(O)ₘR′, CN and N(R″)S(O)ₘR′, provided that two adjacent Rs among the groups R1, R2, R3, R4 and R5 may be an aromatic or cycloalkene ring fused by sharing carbon atoms,
the R′ and R″ are independently one selected from the group consisting of hydrogen, (C1-C10)alkyl, aryl and aryl(C1-C10)alkyl, provided that the R′ and R″ may be a 5-, 6- or 7-membered ring containing 1 to 3 heteroatoms selected from the group consisting of N, O and S since the R′ and R″ are binds to each other if they are connected to the same nitrogen atom, and
m is an integer from 0 to 2.

2. The method according to claim 1, wherein the hydrolysis of S-BBL is carried out in a hydrophilic solvent or a two-phase system of a hydrophilic solvent-hydrophobic organic solvent.

3. The method according to claim 2, wherein the hydrophilic solvent is at least one selected from the group consisting of water, lower alcohol, acetic acid, acetone, ethylacetate, acetonitrile, N,N-dimethylformamide, N,N-dimethylacetamide, dimethylsulfoxide, pyridine, tetrahydrofuran, dioxane, dimethylformamide and dimethylsulfoxide.

4. The method according to claim 2, wherein the hydrophobic organic solvent is at least one selected from the group consisting of isopropyl ether, tert-butyl methyl ether (TBME), chloroform, dichloromethane, carbon tetrachloride, hexane, toluene and cyclohexane.

5. The method according to claim 1, wherein the hydrolase is one selected from the group consisting of lipase, proteinase and esterase.

6. The method according to claim 5, wherein the hydrolase is Candida rugosa-derived lipase, Alcaligenes sp.-derived lipase or their mixture.

7. The method according to claim 1, wherein the hydrolase is an immobilized enzyme.

8. A method for preparing high-purity S-HGB, the method comprising:
(a) preparing S-HGB by hydrolyzing S-BBL represented by the following Formula 1 in the presence of hydrolase: 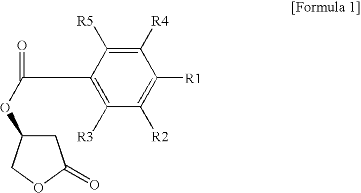
in the Formula 1,
R1, R2, R3, R4 and R5 are independently one selected from the group consisting of hydrogen, (C1-C10)alkyl, (C5-C6)cycloalkyl, fluoro(C1-C10)alkyl, OR′, aryl, aryl(C1-C10)alkyl, NO₂, NR′R″, C(O)R′, CO₂R′, C(O)NR′R″, N(R″)C(O)R′, N(R″)CO₂R′, N(R″)C(O)NR′R″, S(O)ₘNR′R″, S(O)ₘR′, CN and N(R″)S(O)ₘR′, provided that two adjacent Rs among the groups R1, R2, R3, R4 and R5 may be an aromatic or cycloalkene ring fused by sharing carbon atoms,
the R′ and R″ are independently one selected from the group consisting of hydrogen, (C1-C10)alkyl, aryl and aryl(C1-C10)alkyl, provided that the R′ and R″ may be a 5-, 6- or 7-membered ring containing 1 to 3 heteroatoms selected from the group consisting of N, O and S since the R′ and R″ are binds to each other if they are connected to the same nitrogen atom, and
m is an integer from 0 to 2;
(b) removing enzymes from the hydrolysate of the step (a) and extracting benzoic acid in the resultant product with a hydrophobic organic solvent; and
(c) obtaining S-HGB by extracting the benzoic acid-free hydrolysate of the step (b) with a hydrophilic organic solvent at 0° C. or below.

9. The method according to claim 8, wherein the hydrolysis of S-BBL is carried out in a hydrophilic solvent or a two-phase system of a hydrophilic solvent-hydrophobic organic solvent.

10. The method according to claim 9, wherein the hydrophilic solvent is at least one selected from the group consisting of water, lower alcohol, acetic acid, acetone, ethylacetate, acetonitrile, N,N-dimethylformamide, N,N-dimethylacetamide, dimethylsulfoxide, pyridine, tetrahydrofuran, dioxane, dimethylformamide and dimethylsulfoxide.

11. The method according to claim 9, wherein the hydrophobic organic solvent is at least one selected from the group consisting of isopropyl ether, tert-butyl methyl ether (TBME), chloroform, dichloromethane, carbon tetrachloride, hexane, toluene and cyclohexane.

12. The method according to claim 8, wherein the hydrolase is one selected from the group consisting of lipase, proteinase and esterase.

13. The method according to claim 12, wherein the hydrolase is Candida rugosa-derived lipase, Alcaligenes sp.-derived lipase or their mixture.

14. The method according to claim 8, wherein the hydrolase is an immobilized enzyme.

15. The method according to claim 8, wherein the enzyme of the step (b) is removed using a filtration method.

16. The method according to claim 8, wherein the hydrophobic organic solvent of the step (b) is at least one selected from the group consisting of isopropyl ether, tert-butyl methyl ether (TBME), chloroform, dichloromethane, carbon tetrachloride, hexane, toluene and cyclohexane.

17. The method according to claim 8, wherein the hydrophilic organic solvent of the step (c) is at least one selected from the group consisting of methanol, lower alcohol, acetic acid, acetone, ethylacetate, acetonitrile, N,N-dimethylformamide, N,N-dimethylacetamide, dimethylsulfoxide, pyridine, tetrahydrofuran, dioxane, dimethylformamide and dimethylsulfoxide.
